(19) United States
(12) Patent Application Publication
(10) Pub. No.: US 2023/0390254 A1
(43) Pub. Date: Dec. 7, 2023

(54) PHARMACEUTICAL COMPOSITIONS OF UBROGEPANT AND PROCESS FOR PREPARATION THEREOF

(71) Applicant: Aurobindo Pharma Ltd, Hyderabad (IN)

(72) Inventors: Saravanan KANNUSAMY, Hyderabad (IN); Koteswararao GUDE, Hyderabad (IN); Nagaprasad VISHNUBHOTLA, Hyderabad (IN); Sivakumaran MEENAKSHISUNDERAM, Hyderabad (IN)

(21) Appl. No.: 18/204,237

(22) Filed: May 31, 2023

(30) Foreign Application Priority Data
Jun. 3, 2022 (IN) 202241031816

(51) Int. Cl. A61K 31/437; A61K 9/20

(52) U.S. Cl. CPC A61K 31/437; A61K 9/2027; A61K 9/2013; A61K 9/2009; A61K 9/2054; A61K 9/2018; A61K 9/2031

(57) ABSTRACT

The present invention relates to a stable immediate release tablet compositions of Ubrogepant and one or more pharmaceutically acceptable excipients and a process for preparation thereof.

### CROSS REFERENCE TO RELATED APPLICATIONS

This application claims priority from an Indian Patent Application IN 202241031816 filed on Jun. 3, 2022.

### FIELD OF THE INVENTION

The present invention relates to a stable solid oral pharmaceutical compositions of Ubrogepant and pharmaceutically acceptable excipients and a process for preparation thereof.

### BACKGROUND OF THE INVENTION

Ubrogepant is a Calcitonin Gene-related Peptide (CGRP) receptor antagonist. The chemical name is (3′S)—N-((3 S,5S,6R)-6-methyl-2-oxo-5-phenyl-1-(2,2,2trifluoroethyl)piperidin-3-yl)-2′-oxo-1′,2′,5,7 tetrahydrospiro[cyclopenta[b]pyridine-6,3′-pyrrolo[2,3b]pyridine]-3-carboxamide and has the following structural formula:

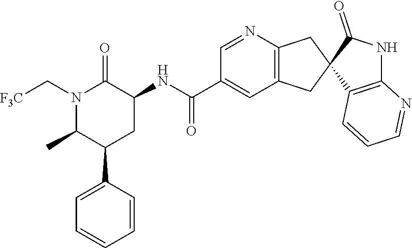 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

The molecular formula is C₂₉H₂₆F₃N₅O₃ and molecular weight is 549.6. Ubrogepant is a white to off-white powder. It is freely soluble in ethanol, methanol, acetone, and acetonitrile; and is insoluble in water. It has been found to be a BCS Class IV compound, which has low solubility and low permeability and difficult to formulate into a solid dosage form for e.g., tablets with desired dissolution and/or bioavailability profiles.

Ubrogepant is commercially available in US market under the brand name UBRELVY® in 50 mg and 100 mg tablets and marketed by Allergan. Ubrogepant is indicated for the acute treatment of migraine with or without aura in adults. The inactive ingredients include colloidal silicon dioxide, croscarmellose sodium, mannitol, microcrystalline cellulose, polyvinyl pyrrolidone vinyl acetate copolymer, sodium chloride, sodium stearyl fumarate and vitamin E polyethylene glycol succinate.

U.S. Pat. No. 8,754,096 discloses Ubrogepant compound.

U.S. Pat. No. 10,117,836 (US′836) discloses that Ubrogepant has poor aqueous solubility and describes compositions comprising an extrudate or solid solution of Ubrogepant in a water-soluble polymer matrix which further comprises a disintegration system allowing a tablet made therefrom to rapidly disintegrate in the environment in which the Ubrogepant is to be released.

The US'836 describes that poorly-soluble Class II compounds are frequently dispersed in many different polymers for the improvement of water solubility. However, in case of Ubrogepant it tends to thermally degrade when attempts are made to incorporate it into a matrix comprising hydroxypropyl methylcellulose acetate succinate (HPMCAS), because it led to the formation of excessive degradation products in the dispersion produced through Hot Melt Extrusion process (HME). In some experiments, use of a cellulosic polymer as the dispersion matrix for preparing dispersions of Ubrogepant by HME resulted in up to 25 times the amount of API degradation that subjecting the API alone to the same thermal excursion generated.

However, inventors of US '836 invented that dispersions or extrudates prepared by HME technique using Ubrogepant, PVP-VA (polyvinylpyrrolidone/vinylacetate) in a polymer matrix of vitamin E in the form of its polyethylene glycol succinate (d-alpha-tocopheryl polyethyleneglycol succinate, or TPGS) with polyethylene glycol 1000; with a disintegration system comprising of croscarmellose sodium and powdered sodium chloride.

Due to at least one of the below reasons, there are greater challenges to design an Ubrogepant oral solid dosage forms, for e.g., tablets with conventionally known pharmaceutically acceptable excipients and/or simple processes:
(a) Poor aqueous solubility of Ubrogepant;
(b) Incompatibility and/or unstable nature of Ubrogepant with certain cellulosic polymers for making solid dispersions using HME process to improve aqueous solubility of Ubrogepant;
(c) No predictability of exploring simple non-HME process of making oral solid dosage forms comprising Ubrogepant;
(d) No predictability of the useful results with the use of water insoluble and/or partially water-soluble polymers;
(e) No predictability of achieving desired dissolution and/or water solubility without using solid dispersions or solid solutions or extrudates of Ubrogepant.

Thus, there is an unmet need to design a desired Ubrogepant oral solid dosage form, like tablets and a simple economical process for producing Ubrogepant tablets having acceptable results of tablet disintegration, tablet dissolution, chemical stability & bioavailability.

### SUMMARY OF THE INVENTION

In one aspect, the present invention provides a stable immediate release tablet comprising of Ubrogepant and a water insoluble polymer selected from the group consisting of hydroxypropyl methylcellulose acetate succinate, hydroxypropyl methylcellulose phthalate and polyvinyl caprolactam-polyvinyl acetate-polyethylene glycol graft copolymer, wherein the tablet is prepared by hot melt extrusion process.

In yet another aspect, the present invention provides a stable immediate release tablet comprising of Ubrogepant, a water-soluble polymer, a dispersing agent, a disintegration system and other pharmaceutically acceptable excipients, wherein the composition is devoid of solid dispersions or solid solutions or extrudates comprising of Ubrogepant.

In another aspect, the present invention provides use of an immediate release tablet of Ubrogepant for the acute treatment of migraine with or without aura in adults.

### DETAILED DESCRIPTION OF THE INVENTION

The present invention relates to a stable immediate release tablet comprising of Ubrogepant, a water insoluble or water soluble polymer and pharmaceutically acceptable excipients and a process for preparation thereof.

The tablet composition of the present invention further comprises, a dispersing agent, a disintegration system, a plasticizer and one or more other pharmaceutically acceptable excipients.

The term “Ubrogepant” as used herein includes “Ubrogepant” per se or its derivatives, solvates, hydrates, enantiomers, isomers, polymorphs, thereof.

According to the present disclosure, Ubrogepant is present in an amount from about 0.5% to about 50% by weight based on the total weight of the composition, preferably Ubrogepant is present in an amount from about 1% to about 20% by weight based on the total weight of the composition.

Examples of water-insoluble polymer include but not limited to hydroxypropyl methylcellulose acetate succinate, hydroxypropyl methylcellulose phthalate, polyvinyl caprolactam-polyvinyl acetate-polyethylene glycol graft copolymer. Water-insoluble polymer is present according to the present invention in an amount from about 1% to about 50% by weight based on the total weight of the composition, preferably, the amount of water-insoluble polymer is from about 2.5% to about 25% by weight based on the total weight of the composition.

Examples of water-soluble polymer include but not limited to polyvinylpyrrolidone-vinyl acetate (PVP-VA) copolymer (copovidone), hydroxypropyl methylcellulose, polyethylene glycol, hydroxypropyl cellulose, carboxymethyl cellulose. Water-soluble polymer is present according to the present invention in an amount from about 1% to about 50% by weight based on the total weight of the composition.

Examples of “dispersing agent” include but not limited to tocopherol-polyethylene-glycolsuccinate (vitamin E TPGS or TPGS), poloxamer, macrogolglycerol hydroxystearate 40, polyoxyethylene castor oils, polyoxyethylene hydrogenated castor oils or combination thereof.

Plasticizers are capable of softening polymers to make them more flexible and lower the processing temperature and thereby decrease the glass transition temperature (Tg) and melt viscosity of the polymer. Examples of “plasticizer” include but not limited to polyethylene glycol 3350, triethyl citrate, or combination thereof.

The term “disintegration system” is a combination of a conventional disintegrant and a rapidly dissolving salt which provides beneficial anti-gellation effects when placed into an environment in which the dosage form within which the disintegration system is incorporated is placed into an environment in which the dosage form disintegrates. For use in a formulation of the present invention, the disintegration system comprises a conventional disintegrant, for example, croscarmellose sodium or crospovidone and sodium chloride. In some embodiments it is preferred for the disintegration system to comprise powdered sodium chloride and croscarmellose sodium, and more preferably in a 1:1 weight ratio. In some embodiments it is preferred for the disintegration system to comprise from about 10% to about 25% by weight based on the total weight of the composition.

The term “pharmaceutically acceptable excipient”, means a pharmacologically inactive component such as a diluent or filler, binder, glidant, lubricant, coloring agent or the like.

Examples of diluent include, but are not limited to microcrystalline cellulose, sodium alginate, silicified microcrystalline cellulose, microfine cellulose, mannitol, maltitol, lactose, anhydrous lactose, lactose monohydrate, spray dried lactose, maltodextrin, isomalt, dicalcium phosphate, dibasic calcium phosphate, tribasic calcium phosphate, sucrose, dextrose, magnesium carbonate and mixtures thereof.

Examples of lubricants include, but are not limited to stearic acid, zinc stearate, sodium stearyl fumarate, magnesium stearate, sucrose stearate, aluminum stearate, adipic acid, carnauba wax, glycerol ester of fatty acid, calcium stearate, glycerol tribehenate, glyceryl behenate, polyethylene glycol and mixtures thereof.

Examples of glidant include, but are not limited to talc, colloidal silicon dioxide, calcium silicate, magnesium silicate, magnesium trisilicate, fumed silica, bentonite, magnesium carbonate, glyceryl monostearate, glyceryl palmitostearate, light anhydrous silicic acid, crystalline cellulose, calcium phosphate tribasic and a combination thereof.

Examples of disintegrating agents include, but are not limited to crospovidone, sodium starch glycolate, croscarmellose sodium, carboxymethyl cellulose sodium, polacrilin potassium and mixtures thereof.

In one embodiment, the present invention provides a stable immediate release tablet comprising:
(a) about 1-20% by weight of Ubrogepant; and
(b) about 2.5-50% by weight of a water insoluble polymer selected from the group consisting of hydroxypropyl methylcellulose acetate succinate, hydroxypropyl methylcellulose phthalate and polyvinyl caprolactam-polyvinyl acetate-polyethylene glycol graft copolymer, wherein the tablet is prepared by hot melt extrusion process.

In another embodiment, the present invention provides a stable immediate release tablet comprising:
(a) about 1-20% by weight of Ubrogepant;
(b) about 2.5-50% by weight of a water insoluble polymer selected from the group consisting of hydroxypropyl methylcellulose acetate succinate, hydroxypropyl methylcellulose phthalate and polyvinyl caprolactam-polyvinyl acetate-polyethylene glycol graft copolymer;
(c) about 1-15% by weight of dispersing agent; and
(d) about 1-15% by weight of plasticizer, wherein the tablet is prepared by hot melt extrusion process.

In one embodiment, the present invention relates to a stable immediate release tablet comprising of:
(a) about 1-20% by weight of Ubrogepant;
(b) about 2.5-50% by weight of water insoluble polymer;
(c) about 1-10% by weight of dispersing agent;
(d) about 0-2.5% by weight of plasticizers;
(e) about 10-50% by weight of diluents;
(f) about 10-25% by weight of disintegration system;
(g) about 0.1-1% by weight of glidant;
(h) about 0.1-2% by weight of lubricant, wherein the tablet is prepared by hot melt extrusion process.

In a particular embodiment, the present invention relates to a stable immediate release tablet comprising of:
(a) about 1-20% by weight of Ubrogepant;
(b) about 2.5-50% by weight of hydroxypropyl methylcellulose acetate succinate;
(c) about 1-10% by weight of tocopherol-polyethylene-glycolsuccinate;
(d) about 0-2.5% by weight of plasticizers selected from polyethylene glycol and triethyl citrate or combinations thereof;
(e) about 10-50% by weight of mannitol and microcrystalline cellulose;
(f) about 10-25% by weight of croscarmellose sodium and sodium chloride;
(g) about of 0.1-1% by weight of colloidal silicone dioxide;
(h) about of 0.1-2% by weight of sodium stearyl fumarate, wherein the tablet is prepared by hot melt extrusion process.

In one embodiment, the present invention relates to a process of making stable immediate release tablet comprising of the following steps:
(a) sift Ubrogepant, a hydroxypropyl methylcellulose acetate succinate, at least one plasticizer selected from polyethylene glycol and triethyl citrate, and at least one diluent selected from mannitol and microcrystalline cellulose through a suitable sieve;
(b) heat tocopherol-polyethylene-glycolsuccinate at 70° C. and add to the step (a) blend;
(c) pass the blend of step (b) through hot melt extruder and get desired extrudates;
(d) dry the extrudates of step (c);
(e) mill the dried extrudates of step (d) to achieve desired granules;
(f) mix the granules of step (e) with extra-granular excipients;
(g) compress the mixture of step (f) into a tablet.

The stable immediate release tablet comprising Ubrogepant according to present invention disintegrates within 15 minutes or preferably between about 1 to 5 minutes.

In yet another embodiment, the present invention provides an immediate release pharmaceutical composition comprising of Ubrogepant, a water-soluble polymer, a dispersing agent, a disintegration system and other pharmaceutically acceptable excipients, wherein the composition is devoid of solid dispersions or solid solutions or extrudates comprising of Ubrogepant. The water-soluble polymer is a polyvinylpyrollidone-vinyl acetate copolymer (PVP-VA), the dispersing agent comprising of tocopherol-polyethylene-glycolsuccinate (TPGS) and the disintegration systems is, for example, croscarmellose sodium (crosslinked sodium carboxymethylcellulose polymer) or crospovidone or combination of croscarmellose sodium/crospovidone and powdered sodium chloride.

In one embodiment, the present invention relates to a stable immediate release tablet comprising of:
(a) about 1-20% by weight of Ubrogepant;
(b) about 2.5-50% by weight of water-soluble polymer;
(c) about 1-10% by weight of dispersing agent;
(d) about 0-2.5% by weight of plasticizers selected from the group of polyethylene glycol and triethyl citrate or combinations thereof;
(e) about 10-50% by weight of diluents;
(f) about 10-25% by weight of disintegration system;
(g) about 0.1-1% by weight of glidant;
(h) about 0.1-2% by weight of lubricant, wherein the composition is devoid of solid dispersions or solid solutions or extrudates comprising of Ubrogepant.

In a particular embodiment, the present invention relates to a stable immediate release tablet comprising of:
(a) about 1-20% by weight of Ubrogepant;
(b) about 2.5-50% by weight of polyvinylpyrollidone-vinyl acetate copolymer;
(c) about 1-10% by weight of tocopherol-polyethylene-glycolsuccinate;
(d) about 0-2.5% by weight of plasticizers selected from polyethylene glycol and triethyl citrate or combinations thereof;
(e) about 10-50% by weight of mannitol and microcrystalline cellulose;
(f) about 10-25% by weight of croscarmellose sodium and sodium chloride;
(g) about of 0.1-1% by weight of colloidal silicone dioxide;
(h) about of 0.1-2% by weight of sodium stearyl fumarate, wherein the composition is devoid of solid dispersions or solid solutions or extrudates comprising of Ubrogepant.

In another embodiment, a process of making stable immediate release tablet comprising of the following steps:
(a) dissolve Ubrogepant, copovidone; tocopherol-polyethylene-glycolsuccinate (TPGS); at least one plasticizer selected from polyethylene glycol and triethyl citrate; in a suitable solvent system selected from Isopropyl alcohol (IPA), ethanol and water or combinations thereof;
(b) spray the solution of step (a) onto the mixture of a diluent & a disintegrant in a top spray granulator;
(c) dry the wet mass of step (b);
(d) mill the dried granules of step (c);
(e) mix the granules of step (d) with extra-granular excipients;
(f) compress the composition of step (e) into a tablet.

In yet another embodiments of the present invention it provides use of a stable immediate release tablet in the manufacture of a medicament for treating migraine with or without aura in adults.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### Example 1: Ubrogepant Tablets

| | | Quantity (mg) | |
|---|---|---|---|
| S. No. | Ingredients | 50 mg | 100 mg |

Intra-granular part

| | | | |
|---|---|---|---|
| 1 | Ubrogepant | 50.00 | 100.00 |
| 2 | Polyvinylpyrrolidone vinyl | 187.5-237.5 | 375.00-475.00 |
| | acetate copolymer | | |
| 3 | Tocopherol-polyethylene- | 12.5-50.00 | 25.00-100.00 |
| | glycolsuccinate | | |
| 4 | Polyethylene glycol/Triethyl | 0.0-12.5 | 0.00-25.00 |
| | citrate/Poloxamer | | |
| 5 | Microcrystalline cellulose | 22.5 | 45.00 |
| 6 | Mannitol | 50.00 | 100.00 |
| 7 | Croscarmellose sodium | 25.00 | 50.00 |
| 8 | Isopropyl Alcohol/Ethanol/ | q.s. | q.s. |
| | Water | | |

Extra-granular part

| | | | |
|---|---|---|---|
| 9 | Microcrystalline cellulose | 22.5 | 45.00 |
| 10 | Mannitol | 50.00 | 100.00 |
| 11 | Croscarmellose sodium | 25.00 | 50.00 |
| 12 | Sodium chloride | 50.00 | 100.00 |
| 13 | Colloidal silicone dioxide | 1.25 | 2.50 |
| 14 | Sodium stearyl fumarate | 3.75 | 7.50 |

| | | |
|---|---|---|
| Total weight | 500.00 | 1000.00 |

### Manufacturing Process:

1. Binder solution preparation: Ubrogepant, copovidone, polyethylene glycol/triethyl citrate/poloxamer, tocopherol-polyethylene-glycolsuccinate were dissolved together in a Isopropyl Alcohol/Ethanol/Water;
2. Granulation: step 1 solution was sprayed onto a mixture of microcrystalline cellulose, mannitol and croscarmellose sodium in a top spray granulator;
3. Drying: the wet granules obtained in step 2 was dried in a suitable dryer until desired LOD got achieved;
4. Milling the dried granules of step 3 were mixed in a comil fitted with a suitable screen;
5. Lubrication: extragranular materials except sodium stearyl fumarate was sifted through a suitable sieve and were mixed for 15 minutes along with milled granules. The above blend was lubricated for appropriate time with sodium stearyl fumarate;
6. Compression: The lubricated blend of step 5 was compressed into tablets.

### Example 2: Ubrogepant Tablets

| | | Quantity (mg) | |
|---|---|---|---|
| S. No | Ingredients | 50 mg | 100 mg |

Intra-granular part

| | | | |
|---|---|---|---|
| 1 | Ubrogepant | 50.00 | 100.00 |
| 2 | Hydroxypropyl methylcellulose | 25.00 | 50.00 |
| | acetate succinate | | |
| 3 | Tocopherol-polyethylene-glycolsuccinate | 20.00 | 40.00 |
| 4 | Polyethylene Glycol 3350 | 20.00 | 40.00 |
| 5 | Crospovidone | 12.85 | 25.70 |
| 6 | Silicon dioxide | 50.00 | 100.00 |
| 7 | Succinic Acid | 75.00 | 150.00 |

Extra-granular part

| | | | |
|---|---|---|---|
| 8 | Silicified Microcrystalline cellulose | 120.407 | 240.815 |
| 9 | Mannitol | 30.50 | 61.00 |
| 10 | Croscarmellose sodium | 45.83 | 91.66 |
| 11 | Sodium chloride | 45.83 | 91.66 |
| 12 | Colloidal silicone dioxide | 1.145 | 2.290 |
| 13 | Sodium stearyl fumarate | 3.438 | 6.875 |

| | | |
|---|---|---|
| Total weight | 500.00 | 1000.00 |

### Manufacturing Process:

1. Ubrogepant, hydroxypropyl methylcellulose acetate succinate, crospovidone, succinic acid, silicon dioxide were co-sifted through appropriate screen;
2. Binder solution of tocopherol-polyethylene-glycolsuccinate & polyethylene Glycol 3350 was prepared by heating;
3. Binder solution of step 2 was added in step 1 mixture to granulate;
4. Step 3 blend was passed through hot melt extrusion and got desired melt extrudates;
5. Extrudates of step 4 were milled through suitable screen;
6. Extragranular materials except sodium stearyl fumarate were sifted through a suitable screen and mixed with milled granules of step 5;
7. The blend of step 6 was then lubricated for appropriate time with sodium stearyl fumarate;
8. The lubricated blend of step 7 was then compressed into tablets.

### In-Vitro Disintegration Study:

The tablet composition comprising Ubrogepant as per the present invention was prepared according to the formula and process of example 2 and the tablets were tested for in-vitro disintegration in a standard disintegration testing apparatus using aqueous HCl (pH 1.8) as a disintegration medium at 37° C. The results of test are mentioned in Table-1 below:

TABLE 1
Disintegration time study results

| | | |
|---|---|---|
| Strength | 50 mg | 100 mg |
| Disintegration Time | 2 min 20 sec | 1 min 25 sec |

### In-Vitro Dissolution Study:

The release profile of the tablet composition comprising Ubrogepant as per the present invention was evaluated through in-vitro dissolution studies. The composition was prepared according to the formula and process of example 2 and stored at accelerated stability conditions (40° C./75% RH for 1.5 month). The initial samples and stability samples were subjected to an in vitro dissolution study in 900 ml of pH 1.8 Simulated Gastric Fluid (Without enzymes) at a temperature of 37±0.5° C. using a USP apparatus-2 (paddle) with peak vessel at a rotation of about 75 rpm in and results are given in Table-2 below. The composition comprising Ubrogepant according to present invention provides comparative dissolution with commercially marketed Ubrogepant tablets (UBRELVY®) at initial and stability condition.

TABLE 2
Dissolution profile of commercially marketed Ubrogepant
tablets (UBRELVY ®) and tablets of example 2:

| | % Ubrogepant released (±5%) | | | | |
|---|---|---|---|---|---|
| Time | UBRELVY ® | 50 mg | | 100 mg | |
| point | 100 mg | | 40° C./75% | | 40° C./75% |
| (Minutes) | Initial | Initial | RH, 1.5M | Initial | RH, 1.5M |
| 5 | 56 | 62 | 57 | 73 | 58 |
| 10 | 97 | 76 | 74 | 86 | 70 |
| 15 | 103 | 85 | 80 | 91 | 79 |
| 20 | 104 | 90 | 86 | 95 | 83 |
| 30 | 104 | 96 | 94 | 98 | 90 |
| 45 | 104 | 99 | 96 | 101 | 95 |
| 60 | 104 | 99 | 98 | 101 | 97 |

### Stability Study:

The stability of the tablet composition comprising Ubrogepant as per the present invention was evaluated through accelerated stability studies. The compositions were prepared according to the formula and process of example 2, and the compositions were subjected to stability study at various temperature and humidity conditions. The compositions were found to be stable at accelerated conditions (40° C./75% RH). Table-3 represents the study result data.

TABLE-3
Stability study results

| | 50 mg | | | 100 mg | | |
|---|---|---|---|---|---|---|
| | | HDPE (H.W) + 1 g silica | | | HDPE(H.W) + 1 g silica | |
| | | gel at 40° C./75% RH | | | gel at 40° C./75% RH | |
| | Initial | 1.5M | 3M | Initial | 1.5M | 3M |
| Total | <0.5% | <0.5% | <0.5% | <0.5% | <0.5% | <0.5% |
| degradation | | | | | | |
| products | | | | | | |
| (Limit: NMT | | | | | | |
| 0.5%) | | | | | | |

We claim:

1. A stable immediate release tablet comprising Ubrogepant and a water insoluble polymer selected from the group consisting of hydroxypropyl methylcellulose acetate succinate, hydroxypropyl methylcellulose phthalate and polyvinyl caprolactam-polyvinyl acetate-polyethylene glycol graft copolymer, wherein the tablet is prepared by hot melt extrusion process.

2. The tablet of claim 1, wherein the tablet further comprises a plasticizer, and one or more pharmaceutically acceptable excipients.

3. The tablet of claim 2, wherein the plasticizer is selected from triethyl citrate and polyethylene glycol.

4. The tablet of claim 2, wherein the tablet further comprises a dispersing agent and a disintegration system.

5. The tablet of claim 4, wherein the dispersing agent is tocopherol-polyethylene-glycolsuccinate and disintegration system is combination of sodium chloride and conventional disintegrants selected from croscarmellose sodium, crospovidone, sodium starch glycolate and low-substituted hydroxypropyl cellulose.

6. The tablet of claim 2, wherein the other pharmaceutically acceptable excipients comprises one or more diluent selected from microcrystalline cellulose, silicified microcrystalline cellulose, microfine cellulose, mannitol, maltitol, lactose and combinations thereof; one or more glidant selected from colloidal silicone dioxide and talc; one or more lubricant selected from magnesium stearate, calcium stearate, stearic acid and sodium stearyl fumarate.

7. The tablet of claim 5, comprising:
(a) about 1-20% by weight of Ubrogepant;
(b) about 2.5-50% by weight of a water insoluble polymer selected from the group consisting of hydroxypropyl methylcellulose acetate succinate, hydroxypropyl methylcellulose phthalate and polyvinyl caprolactam-polyvinyl acetate-polyethylene glycol graft copolymer;
(c) about 1-15% by weight of dispersing agent; and
(d) about 1-15% by weight of plasticizer.

8. A stable immediate release tablet comprising of:
(a) about 1-20% by weight of Ubrogepant;
(b) about 2.5-50% by weight of hydroxypropyl methylcellulose acetate succinate;
(c) about 1-10% by weight of tocopherol-polyethylene-glycolsuccinate;
(d) about 0-2.5% by weight of plasticizers selected from polyethylene glycol and triethyl citrate or combinations thereof;
(e) about 10-50% by weight of mannitol and microcrystalline cellulose;
(f) about 10-25% by weight of croscarmellose sodium and sodium chloride;
(g) about of 0.1-1% by weight of colloidal silicone dioxide;
(h) about of 0.1-2% by weight of sodium stearyl fumarate, wherein the tablet is prepared by hot melt extrusion process.

9. A stable immediate release tablet comprising Ubrogepant, a water-soluble polymer, a dispersing agent, a disintegration system and other pharmaceutically acceptable excipients, wherein the composition is devoid of solid dispersions or solid solutions or extrudates comprising of Ubrogepant, wherein the water-soluble polymer is polyvinylpyrrolidone vinylacetate copolymers.

10. The tablet of claim 9, wherein the dispersing agent is tocopherol-polyethylene-glycolsuccinate.

11. The tablet of claim 9, wherein the disintegration system is combination of sodium chloride and conventional disintegrants selected from croscarmellose sodium, crospovidone, sodium starch glycolate and low-substituted hydroxypropyl cellulose, wherein the other pharmaceutically acceptable excipients comprises one or more plasticizer selected from triethyl citrate and polyethylene glycol or mixtures thereof, a glidant is selected from colloidal silicone dioxide and talc; a lubricant is selected from magnesium stearate, calcium stearate, stearic acid and sodium stearyl fumarate.

12. The tablet of claim 9, wherein the other pharmaceutically acceptable excipients comprises one or more plasticizer selected from triethyl citrate and polyethylene glycol or mixtures thereof, a glidant is selected from colloidal silicone dioxide and talc; a lubricant is selected from magnesium stearate, calcium stearate, stearic acid and sodium stearyl fumarate.
